(19) United States
(12) Patent Application Publication
(10) Pub. No.: US 2010/0216755 A1
(43) Pub. Date: Aug. 26, 2010

(54) ANTI-TUMOR AGENT

(75) Inventors: Yukio NIHEI, Kawasaki-shi (JP); Yoshihiro Morinaga, Kawasaki-shi (JP); Manabu Suzuki, Kawasaki-shi (JP); Yasuyo Suga, Kawasaki-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: AJINOMOTO CO. INC, Tokyo (JP)

(21) Appl. No.: 12/774,912

(22) Filed: May 6, 2010

Related U.S. Application Data
(63) Continuation of application No. 10/743,997, filed on Dec. 24, 2003, now abandoned.
Application No. 10/743,997 is a continuation of application No. PCT/JP02/06260, filed on Jun. 24, 2002.

(30) Foreign Application Priority Data
Jun. 25, 2001 (JP) 2001-191067

(51) Int. Cl. A61K 31/567; A61P 35/00

(52) U.S. Cl. 514/171

(57) ABSTRACT

The present application provides a method for the treatment of tumors where the lethal dose of (Z)—N-[2-methoxy-5-[2-(3,4,5-trimethoxyphenyl)vinyl]phenyl]-L-serinamide is increased to twice or more, the toxicity at the pharmaceutically effective dosage of (Z)—N-[2-methoxy-5-[2-(3,4,5-trimethoxyphenyl)vinyl]phenyl]-L-serinamide is reduced, gastrointestinal toxicity at the pharmaceutically effective dosage of (Z)—N-[2-methoxy-5-[2-(3,4,5-trimethoxyphenyl)vinyl]phenyl]-L-serinamide is reduced, hepatic toxicity at the pharmaceutically effective dosage of (Z)—N-[2-methoxy-5-[2-(3,4,5-trimethoxyphenyl)vinyl]phenyl]-L-serinamide is reduced, and/or cardiovascular toxicity at the pharmaceutically effective dosage of (Z)—N-[2-methoxy-5-[2-(3,4,5-trimethoxyphenyl)vinyl]phenyl]-L-serinamide is reduced, by administering to a subject in need thereof a composition containing:
(a) an effective amount of an anti-inflammatory active substance, wherein the anti-inflammatory active substance is a Dexamethasone selected from the group consisting Dexamethasone, an ester of Dexamethasone, and a salt of Dexamethasone; and
(b) (Z)—N-[2-methoxy-5-[2-(3,4,5-trimethoxyphenyl)vinyl]phenyl]-L-serinamide or a salt thereof.

### CROSS-REFERENCE TO RELATED APPLICATIONS

The present application is a continuation application of International application PCT/JP02/06260, filed on Jun. 24, 2002, which claims priority to Japanese Application No. 2001-191067, filed on Jun. 25, 2001, which are hereby incorporated by reference in their entirety.

### BACKGROUND OF THE INVENTION

1. Field of the Invention

The present invention relates to a novel anti-tumor agent. In particular, the present invention relates to an anti-tumor agent having a combination of a tubulin-polymerization inhibiting active substance, which have anti-tumor activity, and an anti-inflammatory active substance.

The present invention also relates to an anti-tumor pharmaceutical preparation containing one or more tubulin polymerization-inhibitory active substance and/or anti-inflammatory active substance, which can be individually combined with one or more additional tubulin polymerization-inhibitory active substance and/or anti-inflammatory active substance. The present invention also relates to methods and therapeutic regimens for administering the same to a subject in need thereof.

### DISCUSSION OF THE BACKGROUND

Among the anti-tumor agents currently under development relates to medical agents (anti-tumor agents), which contain a tubulin polymerization-inhibitory active substance as an effective component. (Refer to Biochem. Mol. Biol. Int. 25 (6), 1153-1159 (1995); Br. J. Cancer 71 (4), 705-711 (1995); J. Med. Chem. 34 (8), 2579-2588 (1991); Biochemistry 28 (17), 6904-6991 (1989); U.S. Pat. No. 5,561,122; Japanese Laid-Open Patent Application JP-A-07-228,558(1995); Japanese Laid-Open Patent Application JP-A-08-301,831(1996); etc.).

As a result of detailed studies about the medical agent containing a tubulin polymerization-inhibitory active substance, among others, the present inventors have predicted, the possibility of expanding the utility of this medical agent by maintaining the pharmaceutically effective dosage, increasing the lethal dosage and improving the toxicity at the pharmaceutically effective dosage. This possibility arises due to the observation that administration of this medical agent has been restricted in respect to the safety zone (a ratio between the lethal administration dosage and the pharmaceutically effective dosage) and the toxicity at the pharmaceutically effective dosage. Thus, researchers have engaged in the examination for the creation of a medical agent by maintaining the pharmaceutically effective dosage of the effective component in the anti-tumor agent and simultaneously increasing the lethal dosage so that the safety zone for its administration can be expanded, and the toxicity level at the pharmaceutical effective dosage can be reduced, in order to expand its utility as a medical agent, as well as facilitating a reduction on the administration burden on patients and caregivers.

Under the auspices of these observations and objectives, there remains a critical need for the development of an anti-tumor agent which retains the effectiveness (medical effect) of an anti-tumor agent containing a tubulin polymerization-inhibitory active substance as an effective component but which is improved only on the toxicity.

Accordingly, the problem to be solved by the present invention is, when a tubulin polymerization-inhibitory active substance is used as an effective component in an anti-tumor agent, to develop a medical agent (anti-tumor agent) which can maintain its pharmaceutically effective dosage, but at the same time can significantly increase on the lethal dosage and improve toxicity at the pharmaceutically effective dosage of the tubulin polymerization inhibitory active substance.

### SUMMARY OF THE INVENTION

It is an object of the present invention to provide an anti-tumor medical agent (anti-tumor agent) containing a tubulin polymerization-inhibitory active substance in combination with an anti-inflammatory active substance that can significantly increase the lethal administration dosage of a tubulin polymerization-inhibitory active substance and at the same time can maintain its pharmaceutically effective dosage at a level substantially comparable to that for non-combined administration and, as such, the safety zone for the medical agent can be expanded. In addition, it is an object of the present invention to significantly improve the toxicity of the tubulin polymerization-inhibitory substance at the pharmaceutically effective dosage. As a result, the usefulness as an anti-tumor agent to be administered by medical doctors and other caregivers can be remarkably expanded, and the burden to patients can be reduced.

Therefore, the present invention provides an anti-tumor pharmaceutical (pharmaceutical preparation) containing a tubulin polymerization-inhibitory active substance having anti-tumor activity to be used in combination with an anti-inflammatory active substance, and a toxicity reducing agent to be used for an anti-tumor pharmaceutical containing the tubulin polymerization-inhibitory active substance, wherein the agent comprises an anti-inflammatory active substance.

Further, the present invention also provides a method for the anti-tumor, wherein the method includes methods for a medical treatment and an improvement of tumors, a prevention of progression of tumor, and a prevention of tumor; to uses of the above-mentioned 2 effective components for a medical product such as an anti-tumor agent; and to a combination of the above-mentioned 2 effective components wherein the two components are used as a medical product such as an anti-tumor agent, simultaneously or separately, etc.

In order to solve the above-indicated problems in the art, the present inventors have mainly examined combretastatins, stilbenes, derivatives thereof, etc. as a tubulin polymerization-inhibitory active substance that can be utilized as an effective component in such an anti-tumor agent. The inventors have made intensive research works to find out a possible method for retaining the pharmaceutically effective dosage while increasing the lethal dosage, and also for improving various kinds of toxicity caused by administering the pharmaceutically effective dosage, particularly gastroinestinal toxicity, hepatic toxicity and cardiovascular toxicity.

These studies have resulted in a finding that the combined use of an anti-inflammatory active substance, particularly an anti-inflammatory active steroid substance can significantly increase the lethal administration dosage of the tubulin polymerization-inhibitory active substance, favorably to approximately double the original lethal dosage, and can significantly lower toxicity, particularly, its gastroinestinal toxicity, hepatic toxicity and cardiovascular toxicity, while the pharmaceutically effective dosage can be retained substantially at the same level to that without the combined use. As such, the safety zone as a medical agent can be remarkably expanded and the toxicity can be significantly improved within the pharmaceutically effective dosage range.

Namely, the present invention relates to an anti-tumor agent comprising at least a tubulin polymerization-inhibitory active substance having anti-tumor activity and at least an anti-inflammatory active substance (which covers the combination of both the substances) [an anti-tumor agent according to the present invention].

The anti-tumor agent according to the present invention may be in the form of a pharmaceutical (pharmaceutical preparation) containing at least a tubulin polymerization-inhibitory active substance and an anti-inflammatory active substance in a single pharmaceutical (pharmaceutical preparation), or may be in the form of a set of 2 pharmaceuticals (pharmaceutical preparations), for example; an anti-inflammatory agent and an anti-tumor pharmaceutical (pharmaceutical preparation) containing the tubulin polymerization-inhibitory active substance, or in the form of different pharmaceuticals (pharmaceutical preparations) wherein 2 kinds of the pharmaceuticals (pharmaceutical preparations) are used in combination.

The tubulin polymerization-inhibitory active substance may be any substance as far as it has tubulin polymerization-inhibitory activity, and there is no other particular restriction. It is necessary to select a substance which has anti-tumor activity, but any such a substance may be adopted without restriction, either it is a known substance or a substance to be developed in future. For example, such a tubulin polymerization-inhibitory active substance may be selected from the group consisting of combretastatines and derivatives thereof, vinca alkaloids such as vinblastine, etc. and derivatives thereof, colchicines and derivatives thereof, dolastatins and derivatives thereof, podophyllotoxins and derivatives thereof, steganacins and derivatives thereof, amphethiniles and derivatives thereof, flavonoids and derivatives thereof, rhizoxins and derivatives thereof, curacins A and derivatives thereof, epothilones A and derivatives thereof, epothilones B and derivatives thereof, welwistatins and derivatives thereof, phenstatins and derivatives thereof, 2-strylquinazoline-4(3H)-ones and derivatives thereof, stilbenes and derivatives thereof, 2-aryl-1,8-naphthyridin-4(1H)-ones and derivatives thereof, 5,6-dihydroindolo(2,1-a) isoquinolines and derivatives thereof, 2,3-benzo(b)thiophenes and derivatives thereof; 2,3-substituted benzo(b)furans and derivatives thereof, 2,3-substituted indoles and derivatives thereof, and 2-methoxyestradiol (refer to the WO 00/48606 Specification).

Various reports exist reporting that combretastines, stilbenes and derivatives thereof have anti-tumor activity (refer to J. Med. Chem. 41: 3022-3032 (1998); Bioorg. Med. Chem. Lett. 8:3153-3158 (1998); Bioorg. Med. Chem. Lett. 8:3371-3374 (1998); U.S. Pat. No. 5,561,122; U.S. Pat. No. 5,430,062; Japanese Laid-Open Patent Application JP-A-07-228,558(1995); Japanese Laid-Open Patent Application JP-A-08-301,831(1996), the WO93/23,357 specification, the WO99/51,246 specification; etc.), and those substances described therein can be utilized within the present invention (and all the descriptions about these tubulin polymerization-inhibitory active substances are included in the present specification by reference). As a typical example of the derivatives, (Z)—N-[2-methoxy-5-[2-(3,4,5-trimethoxyphenyl)vinyl]phenyl]-L-serinamide hydrochloride (hereinafter called “AC-7700) may be cited.

In addition, all the substances cited in the specification of WO 00/48,606 as having tubulin polymerization-inhibitory activity can be used as a tubulin polymerization-inhibitory active substance according to the present invention, and all the substances cited to have tubulin polymerization-inhibitory activity, any descriptions about these substances in the international patent publication (specification) or in prior art publications cited therein (as related descriptions) are incorporated herein by reference.

No particular restriction exists on the anti-inflammatory active substance to be used according to the present invention, but it can be favorably selected from the group consisting of anti-inflammatory active steroid substances and analogous substances thereof, anti-inflammatory active non-steroid substances and analogous compounds thereof, and anti-inflammatory or immuno-suppressive active substances.

The anti-inflammatory active substance to be used according to the present invention may be preferably an anti-inflammatory active steroid substance.

Such an anti-inflammatory active substance may be selected from the group consisting of Dexamethasone and derivatives thereof, prednisolone and derivatives thereof, methylprednisolone and derivatives thereof, betamethasone and derivatives thereof, triamcinolone and derivatives thereof, paramethasone and derivatives thereof, beclomethasone and derivatives thereof, flucinolone acetonide and derivatives thereof and cortisol (natural glucocorticoid) and derivatives thereof.

It can be favorably selected from the group consisting of Dexamethasone and derivatives thereof, particularly Dexamethasone, phosphate ester thereof and salts thereof (such as sodium salt, etc.). Conveniently, a commercial distributed anti-inflammatory agent can be procured for the use on the market.

As typical examples for the 2 kinds of the effective components, the tubulin polymerization-inhibitory active substance may be selected particularly from the group consisting of combretastatins and derivatives thereof and stilbenes and derivatives thereof, and the anti-inflammatory active substance may be also selected from the group consisting of Dexamethasone and derivatives thereof (ester, etc.).

A pharmaceutical (pharmaceutical preparation) containing the tubulin polymerization-inhibitory active substance (an anti-tumor pharmaceutical (pharmaceutical preparation)) and a pharmaceutical (pharmaceutical preparation) containing an anti-inflammatory active substance (an anti-inflammatory agent) can be simultaneously administered or can be administered at different times. Accordingly, the pharmaceutical preparations may be in the form for respective administrations or in the form of a single pharmaceutical (pharmaceutical preparation). Both the pharmaceuticals may be in different forms for respective administration, and in this case, both the pharmaceuticals have to constitute respectively different pharmaceuticals.

The tubulin polymerization-inhibitory active substance can be used in the form of an anti-tumor pharmaceutical (pharmaceutical preparation; pharmaceutical agent) and the anti-inflammatory active substance can be used in the form of an anti-inflammatory agent.

In another object, the present invention relates to an anti-tumor pharmaceutical (pharmaceutical preparation; pharmaceutical agent) containing a tubulin polymerization-inhibitory active substance having anti-tumor activity, wherein the anti-tumor pharmaceutical is to be used in combination with or as a set with an anti-inflammatory active substance (an anti-tumor pharmaceutical (pharmaceutical preparation; pharmaceutical agent) according to the present invention).

According to further another object, the present invention relates to a toxicity-reducing agent to be used for an anti-tumor pharmaceutical (pharmaceutical preparation; pharmaceutical agent) containing a tubulin polymerization-inhibitory active substance, wherein the toxicity-reducing agent comprises an anti-inflammatory active substance (a toxicity-reducing agent according to the present invention).

The present invention provides an anti-tumor agent, but it also provides 2 different objects. Those agents according to the 2 different objects of invention are substantially the same with the anti-tumor agent according to the present invention in a sense that both the 2 different objects of invention are related to a medical agent wherein 2 kinds of effective components: a tubulin polymerization-inhibitory active substance having anti-tumor activity and an anti-inflammatory active substance are combined, and therefore, can be easily worked based on explanations about the anti-tumor agent according to the present invention. The term of “an anti-tumor pharmaceutical (pharmaceutical preparation; pharmaceutical agent)” is a term used for its differentiation from the “anti-tumor agent” according to the present invention, and any and all medical agents containing the tubulin polymerization-inhibitory active substance and used for the therapy, betterment or other treatments of tumors are equally and completely covered under the term, regardless of whatever names those medical agents are termed, including anti-tumor agents, etc.

According to the present invention, an anti-tumor medical agent can be, for example, a combination of the 2 kinds of the effective components in respectively different pharmaceutical forms, and moreover in the forms which can be administered at the same time or different times. Also, these 2 effective components may be included in the same pharmaceutical (pharmaceutical preparation; pharmaceutical agent) that allows individual administration by pharmaceutical units. Even in such objections of the present invention, the anti-tumor agent according to the present invention also covers medical agents containing at least the 2 kinds of the effective components.

Consequently, it is not necessary that plural pharmaceuticals respectively containing at least the 2 kinds of the effective components to be used according to the present invention are jointly and fixedly contained in a specific package or container, and even when these pharmaceuticals are respectively and independently present, these pharmaceuticals shall be covered by the anti-tumor agent according to the present invention as far as these pharmaceuticals are used for the same purpose as according to the present invention (the realization of an anti-tumor effect).

Furthermore, although it is indispensable that the anti-tumor agent according to the present invention contains the 2 kinds of the effective components which are used in an appropriate combination, further different effective components [components having the same medical effect (anti-tumor components) or components having a different medical effect, components for enhancing an intended medical effect, components for the further reduction of toxicity (a side effect), and other components] may be used for combination or inclusion in pharmaceuticals according to the present invention, as far as these additional components do not hinder the effect of the present invention. In the preparation of pharmaceuticals, additive components required can be appropriately selected and used for the pharmaceutical preparation.

According to another object of the present invention is a method for the anti-tumor, which comprises administering containing a tubulin polymerization-inhibitory active substance having anti-tumor activity and an anti-inflammatory active substance to subject in need thereof. The method includes methods for a medical treatment and an improvement of tumors, a prevention of progression of tumor, and a prevention of tumor. These 2 kinds of effective components can be administered to a living body at the same time, or separately at different times. The administration form as such can be selected from various forms in the above-mentioned anti-tumor agent according to the present invention, the anti-tumor pharmaceutical preparation, and the toxicity-reducing agent.

According to yet another objection of the present invention is a use of a tubulin polymerization-inhibitory active substance having anti-tumor activity and an anti-inflammatory active substance for a medical product such as an anti-tumor agent. The tubulin polymerization-inhibitory active substance having anti-tumor activity and the anti-inflammatory active substance are can be used individually in the different pharmaceutical preparation forms. The form for uses in the medical product as such can be selected from various forms in the above-mentioned anti-tumor agent according to the present invention, the anti-tumor pharmaceutical preparation (agent), and the toxicity-reducing agent.

In still another objection of the present invention is a combination of a tubulin polymerization-inhibitory active substance having anti-tumor activity with an anti-inflammatory active substance wherein the two substances are used as a medical product such as an anti-tumor agent, simultaneously or separately.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1: FIG. 1 shows results from the toxicity test with tumor-bearing rats from Example 1 (Scheffe's F test; *p<0.05, **p<0.01)

F344 rats subcutaneously transplanted MT-9 tumor/Dexamethasone (1 mg/kg)/AC-7700 (10 mg/kg); Blood biochemical indices: GPT; ▪:−DEX; □:+DEX.

FIG. 2: FIG. 2 shows results from the toxicity test with tumor-bearing rats in Example 1 (Scheffe's F test; *p<0.05).

F344 rats subcutaneously transplanted MT-9 tumor/Dexamethasone (1 mg/kg)/AC-7700 (10 mg/kg); Blood biochemical indices: CPK; ▪:−DEX; □:+DEX.

### DETAILED DESCRIPTION OF THE INVENTION

Unless specifically defined, all technical and scientific terms used herein have the same meaning as commonly understood by a skilled artisan in biochemistry, cellular biology, molecular biology, and the medical sciences, in particular oncology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

In the following, the present invention shall be explained mainly on the mode as an anti-tumor agent according to the present invention, but other modes of the present invention can be similarly understood based on this explanation because the same 2 kinds of medical components: a tubulin polymerization-inhibitory active substance having anti-tumor activity and an anti-inflammatory active substance may be combined in all the modes for applying the present invention.

In one embodiment of the present invention, the anti-tumor agent is a medical agent which contains (a) at least said 2 kinds of effective components, (b) plural components being capable to be administered at the same or different times, (c) the 2 kinds of components being administered in the form of a same pharmaceutical or (d) different pharmaceuticals for combined use for the broad purpose of tumor suppression, such as the betterment, prevention, therapy, growth inhibition, etc. of tumors in mammals. In a particular embodiment the mammal is a human.

As described above, as far as this effect can be achieved, it can additionally contain or be combined with other medical components, and it can also contain other components that are required for pharmaceutical preparation.

Within the context of the present application, the tubulin polymerization-inhibitory active substance may be selected from known tubulin polymerization-inhibitory active substances having anti-tumor activity (refer to the above cited prior art publications), as well as the stilbene derivatives described below.

Examples of the stilbene derivatives to be used in the present invention may include compounds having the fundamental skeleton of cis-stilbene, which exhibit the in-vitro activities of tubulin polymerization inhibition and anti-tumor activity. As for anti-tumor activity, those compounds particularly exhibiting the activity of tumor cell growth inhibition are preferable. Any of the stilbene derivatives, either known or to be found in future, should be included in the stilbene derivatives to be used according to the present invention. Moreover, the stilbene derivatives shall include derivatives that are converted to a stilbene derivative within the body of an animal to which the parent compound has been administered. As far as a stilbene derivative can exhibit the intended anti-tumor activity according to the present invention when used within the body of an animal to which the parent compound has been administered, and may it be a salt, ester, solvate such as hydrate, etc., as far as it is a pharmaceutically acceptable derivative, it can be used as a stilbene derivative having tubulin polymerization-inhibitory activity according to the present invention.

Typical stilbene derivatives having the fundamental skeleton of cis-stilbene include compounds preferably represented by the following formulae (1) and (2). These compounds include those in the form of various salts, hydrates and solvates, and particularly those in pharmaceutically acceptable forms.

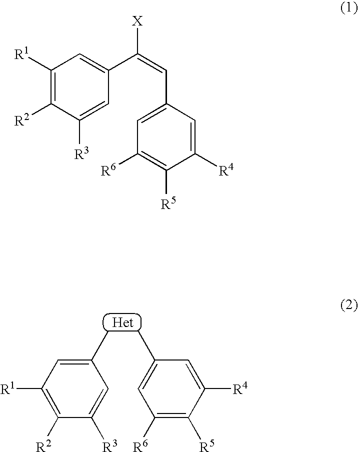

wherein R¹, R² and R³ respectively and independently denote a lower alkoxy group, R⁴, R⁵ and R⁶ respectively and independently denote a hydrogen atom, a halogen atom (fluorine, chlorine, etc.), any of substitution groups including nitro, hydroxy, lower alkoxy, phosphate ester (a substitution group formed by phosphoric ester formation with a hydroxy group: —OP₃H₂, hereunder meaning the same), phosphate amide (a substitution group formed by phosphoric amide formation with an amino group: —NHPO₃H₂, hereunder meaning the same), amino lower alkoxy, lower alkyl amino lower alkoxy, di-lowera alkyl amino lower alkoxy, mercapto, lower alkyl thio, amino, lower alkyl amino, di-lower alkyl amino, lower alkyl, amino lower alkyl, trifluoromethyl, lower alkanoyl, lower alkanoyl amino and amino acid acyl amino, X denotes a hydrogen atom or a nitrile group, and Het denotes a heterocycle.

The lower alkyl and lower alkoxy groups are respectively to have 1-5 carbon atoms. Also, the lower alkanoyl group is to have 2-6 carbon atoms.

An amino acid acyl group in the amino acid acyl amino group is an acyl group derived from an amino acid, and examples of the amino acid may include α-amino acid, β-amino acid and γ-amino acid. Preferable examples of the amino acid may include glycine, alanine, leucine, serine, lysine, glutamic acid, aspartic acid, threonine, valine, isoleucine, ornithine, glutamine, asparagine, tyrosine, phenyl alanine, cystine, methionine, alginine, β-alanine, tryptophan, proline, histidine, etc. Particularly preferable in respect of the pharmaceutical effect and safety are threonine and serine. These amino acids may be of any of L-, D- and DL-forms, but the L-form is preferable.

Examples of the heterocycle may include a tetrazole ring, a thiazole ring, etc. When the heterocycle is a thiazole ring, it may be substituted. Examples of a substitution group in this case may include lower alkyl, amino, mono-lower alkyl amino, di-lower alkyl amino, hydrazino, halogen atom (fluorine, chlorine, etc.) and lower alkoxy. Incidentally, the lower alkyl and love er alkoxy groups are respectively to have 1-5 carbon atoms.

As described above, a stilbene derivative to be used according to the present invention is structurally a compound having a cis-stilbene skeleton, and a compound exhibiting tubulin polymerization-inhibitory activity and anti-tumor activity. As a specific example of the stilbene derivative, combretastatin-A4 may be cited, but the stilbene derivative should not be particularly restricted, and may include any stilbene derivatives capable of inhibiting tumor growth, which have been disclosed in prior art publications, for example, patent gazettes, etc. (refer to U.S. Pat. Nos. 4,996,237; 5,561,122 and 5,430,062; and Japanese Laid-Open Patent Applications JP-A-07-228,558(1995); JP-A-08-301,831(1996) and JP-A-10-81,673(1998); etc.). Stilbene derivatives described in these prior art publications can be utilized as stilbene derivatives according to the present invention as far as those are covered under the above stated definitions, and as described above, those disclosures in the prior art publications are included as a part constituting the present patent specification.

When the stilbene derivatives are to be manufactured, these compounds can be manufactured according to known technology including manufacturing processes disclosed in the above described publications. Stilbene derivatives to be developed in future may be also manufactured and utilized in the same manner as described above.

Stilbene derivatives to be used according to the present invention may include those in the form of salts, esters, solvates such as hydrates and other derivatives, as well as derivatives which may be converted in animal body to stilbene derivatives as far as those derivatives can exhibit said activity in an animal body to which the compound is administered.

More preferable stilbene derivatives to be used according to the present invention are represented by the following formula (1′):

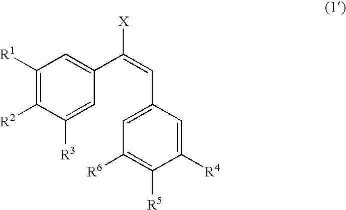

In the formula (1′), R¹, R², R³ and R⁵ respectively denote a methoxy group, R⁴ denotes either an amino group or an amino acid acyl amino group, and R⁶ and X respectively denote a hydrogen atom.

Among compounds represented by the formula (1′), a compound represented by the formula (3) [(Z)—N-[2-methoxy-5-[2-(3,4,5-trimethoxyphenyl)vinyl]phenyl]-L-serinamide] is particularly preferable [which is hereunder called Compound (3)]. The Compound (3) may be in the form of a salt, which may include hydrochloride (AC-7700), acetate, methane sulfonate, etc.

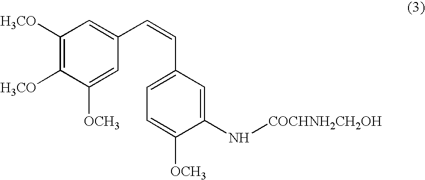

The manufacture of Compound (3) (including pharmaceutically acceptable salts, hydrates and solvates thereof) as well as the manufacture of orally or non-orally administered pharmaceutical compositions containing Compound (3) and an inert, pharmaceutically acceptable carriers or diluent have been disclosed in Japanese Laid-Open Patent Application JP-A-08-301,831(1996) in a broad range, which can be referred to for the manufacture thereof.

The compound may be used, as a medical agent according to the present invention, in the form of a pharmaceutical (pharmaceutical preparation) that contains a tubulin polymerization-inhibitory active substance having anti-tumor activity. In this case, it can be used in the form of a single pharmaceutical (pharmaceutical preparation) containing the tubulin polymerization-inhibitory active substance as a (major) effective component, or it can be also used in the form of a pharmaceutical (pharmaceutical preparation) that combines the anti-inflammatory active substance with this component (a pharmaceutical combining 2 kinds of effective components).

There is no particular restriction on the form of pharmaceutical preparations in this case. It can be used in the form of an orally administered pharmaceutical or a non-orally administered pharmaceutical, particularly in the form of an injection. Investigations for their use as an anti-tumor agent have been already under way on some of the tubulin polymerization-inhibitory active substances (for example, combretastatines, vinca alkaloids, colchicinoids, dolastatins, podophyllotoxins, rhizoxins, 2-methoxyestradiol, etc.), and thus, their pharmaceuticals (pharmaceutical preparations) can be easily prepared based on public knowledge obtained in relation to such investigations.

An anti-inflammatory active substance to be used in the anti-tumor agent according to the present invention can be combined and used in said pharmaceuticals, but what has been known and used as an anti-inflammatory agent can be separately used for the combined use with a pharmaceutical, which contains the tubulin polymerization-inhibitory active substance having anti-tumor activity. It can be used according to a known administration method, etc. for an anti-inflammatory agent. Due to a component to be combined for use or the combined use of an anti-inflammatory active substance according to the present invention, the lethal dosage of the tubulin polymerization-inhibitory active substance is increased, preferably to about twice or more, and remarkable improvement on the toxicity at the pharmaceutically effective dosage can be achieved, particularly on gastrointestinal toxicity (betterment of diarrhea, etc.), hepatic toxicity (lowering of GPT, etc.) and cardiovascular toxicity (lowering of CPK, etc.). On the other hand, it has been found that the pharmaceutically effective dosage has not been affected by the presence or absence of the combined use according to the present invention.

Accordingly, the present invention provides therapeutic regimens in which the tubulin polymerization-inhibitory active substance may be administered concurrent with or in addition to an anti-inflammatory active substance.

Further, when a tubulin polymerization-inhibitory active substance is used as an anti-tumor agent, a remarkable improvement can be achieved on the lethal toxicity and the toxicity at the pharmaceutically effective dosage, so that medical people such as doctors, et al can conveniently use this medical agent, and the burden on patients administered of this medical agent can be greatly reduced, too.

For example, depending upon symptoms of patients, age, physical condition, etc., the dosage of a tubulin polymerization-inhibitory active substance, for example in the case of AC-7700 (an injection) can be ascertained. In a preferable embodiment, the range of the dosage to be administered to the subject in need thereof of a tubulin polymerization-inhibitory active substance is about 0.1-10000 mg, more preferably about 0.5-1000 mg, and further preferably about 1-500 mg per day. The dosage to be administered to the subject in need thereof of an anti-inflammatory active agent to be used in combination according to the present invention, for example in the case of Dexamethasone sodium phosphate (an injection), can be preferably about 0.1-10000 mg, more preferably about 0.5-1000 mg and further preferably about 1-500 mg per patient a day. In each of the ranges set forth above, it is to be understood that the dosage amount may be in a unit dosage form or may be administered several times in equal or scaled concentrations over the course of the day.

When these agents are to be orally administered, respective dosages can be within a range of about 2-20 times the dosage used as an injection.

The dosage for the administration of the 2 kinds of effective components according to the present invention together with other components or as derivatives thereof can be optionally selected in utilization of prior art technology and measures, by referring to the above-described ranges of the dosages.

The 2 kinds of components as indispensable effective components to be used according to the present invention can be respectively contained in different pharmaceutical forms and independently administered to patients who desire an anti-tumor effect. But as described above, these 2 components can contain or be combined with other pharmaceutical components to be used as a medical agent exhibiting an anti-tumor effect, and these cases are also naturally covered by the present invention as far as these medical agents exert an anti-tumor effect. A tubulin polymerization-inhibitory active substance can be combine with an anti-inflammatory active substance for medical uses, and in this case, further components can be contained or be combined with these 2 kinds of components for the use of a tubulin polymerization-inhibitory active substance so that a similar anti-tumor effect can be exerted, and such a use is also covered by the present invention.

In the preparation of pharmaceuticals, pharmacologically acceptable, various preparatory substances can be also contained (as supplemental agents, etc.). Substances for pharmaceutical preparation can be optionally selected depending upon the form of preparations, examples of which may include vehicles, diluents, additives, disintegrators, binders, coating agents, lubricants, sliding agents, smoothing agents, flavoring agents, sweeteners, solubilizers, etc. Furthermore, specific examples of preparatory substances may include magnesium carbonate, titanium dioxide, lactose, mannitol and other saccharides, talc, milk casein, gelatin, starch, cellulose and derivatives thereof, animal and vegetable oils, polyethylene glycol, and solvents such as sterile water and mono- or poly-hydric alcohols, for example, glycerol, etc.

It is not necessary to include all the effective components to be used according to the present invention in the same single pharmaceutical and respective components or the 2 components may be appropriately contained in one or two pharmaceuticals. In this case, the components may be prepared to various forms of pharmaceuticals, either known or to be developed in future, for example, including various administration forms such as for oral administration, abdominal administration, dermal administration, inhalation administration, intravenous administration, etc. In order to prepare pharmaceutical components to be used according to the present invention into such various pharmaceutical forms, methods, either known or to be developed in future, may be optionally adopted.

Examples of such various forms of pharmaceuticals may include appropriate solid or liquid pharmaceutical forms, such as granules, powder, coated tablets, tablets, (micro-) capsules, depositories, syrups, juices, suspensions, emulsions, drops, injection solutions, preparations for extended release of an active substance, etc.

Medical agents according to the present invention, prepared in pharmaceutical forms such as cited above in the examples, should naturally contain pharmaceutically effective amounts of said components in order to achieve the intended effect.

### (Anti-Tumor Pharmaceutical According to the Present Invention)

As described above, an anti-tumor pharmaceutical containing a tubulin polymerization-inhibitory active substance having anti-tumor activity, which is characterized by the combined use with an anti-inflammatory active substance, is provided by the present invention. Since this pharmaceutical is substantially the same with the anti-tumor agent according to the present invention in respect to the combined use of the 2 kinds of medical components, a skilled person in the art should be able to practice this invention based on the above described detailed explanation and later described examples, as well as prior art technology.

### (Toxicity-Reducing Agent According to the Present Invention)

Similarly, a toxicity-reducing agent for an anti-tumor pharmaceutical containing a tubulin polymerization-inhibitory active substance, wherein an anti-inflammatory active substrate is contained, is also provided by the present invention. Since this toxicity-reducing agent is substantially same with the anti-tumor agent according to the present invention in respect to the combined use of the 2 kinds of medical components in the same manner as described above, a skilled person in the art should be able to practice this invention based on the above described detailed explanation and later described examples, as well as prior art technology.

As described above, according to other modes, the present invention provides the following:

i) A method, which comprises administering to a subject in need thereof a to composition containing a tubulin polymerization-inhibitory active substance having anti-tumor activity and an anti-inflammatory active substance, wherein the method includes methods for a medical treatment and an improvement of tumors, a prevention of progression of tumor, and a prevention of tumor;

ii) A tubulin polymerization-inhibitory active substance having anti-tumor activity and an anti-inflammatory active substance for a medical product such as an anti-tumor agent; and

iii) A combination of a tubulin polymerization-inhibitory active substance having anti-tumor activity with an anti-inflammatory active substance wherein the two substances are used as a medical product such as an anti-tumor agent, simultaneously or separately.

The embodiments of the present invention may all be carried out readily on the basis of the descriptions about the above-mentioned anti-tumor agent, anti-tumor pharmaceutical preparation (agent), and/or toxicity-reducing agent according to the present invention or the after described Examples and the like, with reference to known art, if necessary.

Having generally described this invention, a further understanding can be obtained by reference to certain specific examples, which are provided herein for purposes of illustration only, and are not intended to be limiting unless otherwise specified.

### Examples

### Example 1

### (1) Tumor Cell Line and Experimental Animal

Rat tumor cell line (transplant rat strain);
Malignant fibrous histiocytoma MT-9 (F344, male)

MT-9 was obtained as in-vitro cultured cells, which were cultured in RPMI1640 medium containing 10% FBS. 10⁷ or more of the MT-9 cells were subcutaneously transplanted into the back of rats. After tumor formation, tumor slices (about 100 mg) were inoculated subcutaneously into rats using cannula for serial passage.

F344 (5 weeks old) was acquired from Charles River Japan.

### (2) Medicines and Administration Method

(Z)—N-{2-methoxy-5-[2-(3,4,5-trimethoxyphenyl)vinyl]phenyl]-L-serinamide hydrochloride (AC-7700) was employed as the tubulin polymerization-inhibitory active substance having anti-tumor activity.

AC-7700 was stored in a dark place at a low temperature (5° C.) after the synthesis. After weighing, AC-7700 was dissolved in physiological saline immediately prior the administration.

As the Dexamethasone (its derivative), “Decadron S injection” made by Banyu Pharmaceutical (Demethasone sodium phosphate, its chemical term being 16α-methyl-9α-fluoroprednisolone 21-phsophate disodium salt, hereinafter simply called “Dexamethasone” or “DEX”) was diluted with physiological saline immediately prior to the administration by intravenous injection.

### (3) Procedure for the Biochemical Testing of Blood

Under ether anesthesia, rats were subjected to ventrotomy, and blood was sampled from inferior vena cava using heparin-containing syringes. The blood samples were centrifuged at 3000rpm for 10 minutes to recover plasma, and the GOT, GPT, CPK and LDH concentration in plasma were determined using Fuji Dry Chem. When the measurement was not conducted immediately, the plasma samples were stored at −80° C. until the measurement was conducted.

(4) Procedure for the Evaluation of in vivo Pharmaceutical Effect

### (Anti-Tumor Effect)

The MT-9/F344 system: Tumors serially passed by the subcutaneous transplantation were extirpated. After binding tissues and necrosis portions in the tumors had been removed, tumor tissues were minced with scissors and made pasty. About 50-100 mg of the tumor tissues were subcutaneously transplanted into the backs of F344 rats (day 0).

After the tumors were multiplied to a measurable quantity (about 1-2 weeks later), the tumor size (tumor volume) and body weight were measured and divided into tumor size-and body weight-matched groups.

The tumor sizes (tumor volumes) and body weights were measured every day from the next day to about the third day following completion of administration. Incidentally, the tumor volume was calculated according to the following formula:

Tumor volume (mm³)=[(larger diameter, mm)×(smaller diameter, mm)]²×½.

For the determination of the anti-tumor effect, the T/C and I.R. values were calculated according to the following formula, and when the T/C value was 50% or less (the I.R. value being 50% or more) and statistically significant difference from the value for control existed, the anti-tumor effect was judged to be positive, and the anti-tumor effect on a day when the maximum anti-tumor effect was observed was determined as the pharmaceutical effect.

T/C(%)=[(tumor volume for the medicine administered group)÷(tumor volume for the control group)]×100

I.R.(%)=100−T/C.

Weight changes were derived by the deduction of the body weight on the day when the treatment was started from the body weight on the day for evaluating the pharmaceutical effect. Incidentally, the tumor weight (g) was calculated by conversion as (tumor volume× 1/1000), and changes in the body weight excluding the tumor weight were determined based on the deduction of respective tumor weights from the body weight.

### (5) Statistical Analysis

The growth of rat tumors was statistically analyzed on the presumption that it does not follow normal distribution. In the comparison between the control group and treated groups administered with respective dosages (2 group comparison), the Mann-Whitney U test was used, and a P value of 0.5 or less was judged to be significant. In the multiple group comparison, Scheffs F test was used, and a P value of 0.05 or less was judged to be significant.

### (6) Results

(1) Reduction of AC-7700 Toxicity with Dexamethasone

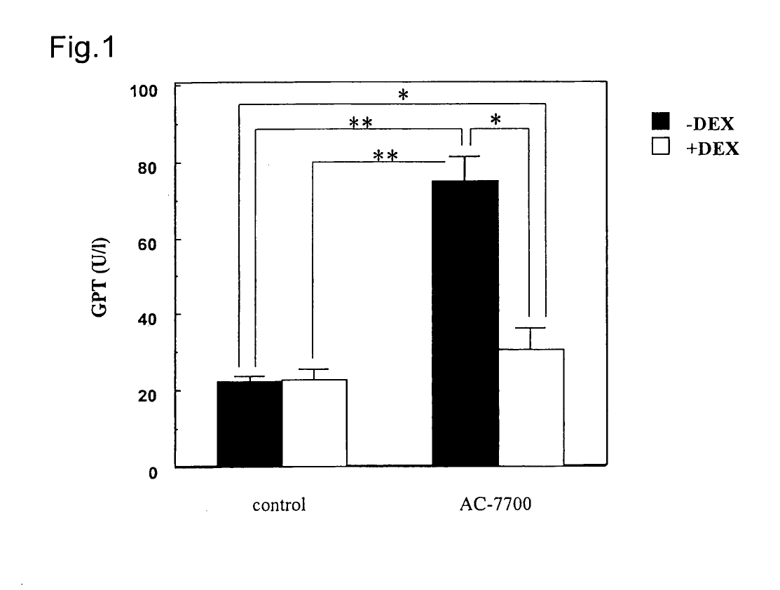

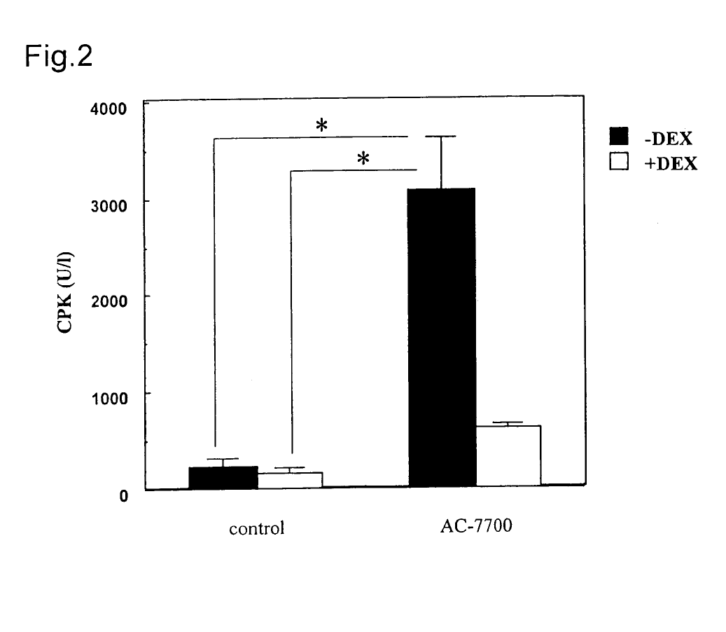

MT-9 was subcutaneously transplanted into the backs of F344 rats, and after the tumor was formed, the medical agent was administered. AC-7700 was administered at a pharmaceutically effective single agent dosage of 10 mg/kg, and Dexamethasone was administered at a dosage of 1 mg/kg on a day before the administration of AC-7700. The biochemical indices of blood were measured 6 hours after AC-7700 administration. Results are shown in FIGS. 1 and 2.

The results reveal that Dexamethasone had remarkably reduced the toxicity of AC-7700 (10 mg/kg), hepatic toxicity (GPT) and cardiovascular toxicity (CPK) in tumor bearing rats.

Concerning the gastrointestinal toxicity, the combined use of Dexamethasone with AC-7700 has revealed that diarrhea induced by AC-7700 in mice was significantly improved. (2) Influence of Dexamethasone on the pharmaceutical effect of AC-7700

The influence of Dexamethasone on the pharmaceutical effect of AC-7700 was investigated using F344 rats, subcutaneously transplanted MT-9. AC-7700 was administered at a dosage of 10 mg/kg, and 1 mg/kg of Dexamethasone was administered on the day before the AC-7700 administration. Both the medical agents were 3 times administered every 3 days.

AC-7700 significantly inhibited tumor growth in a single agent. The administration of AC-7700 in combination with Dexamethasone also inhibited tumor growth in the same manner (refer to Table 1). There was no significant difference between anti-tumor effect of AC-7700 and those of AC-7700 combined with Dexamathasone (Scheffe's F test).

TABLE 1
Influence of Dexamethasone on the
pharmaceutical effect of AC-7700

| DEX(mg/kg/day) | AC-7700(mg/kg/day) | I.R.(%) |
|---|---|---|
| 0 | 0 | 0 |
| 0 | 10 | 84** |
| 1 | 0 | 21 |
| 1 | 10 | 72** |

(Note: Mann-Whitney's U test; **p < 0.01 vs. Control)

Furthermore, in the investigation using CDF1 mice (female), Dexamethasone (5 mg/kg/day administered by intravenous injection into the tail, on a day before and on the day of the AC-7700 administration), increased the maximum tolerable dose of AC-7700 (3 times subcutaneously administered every 3 days) from 43.6 mg/kg/day to more than 90 mg/kg/day.

### Advantages of the Invention

According to the present invention, an anti-tumor agent containing a tubulin polymerization-inhibitory active substance as an effective component is provided, wherein the medical agent (anti-tumor agent) in combination with an anti-inflammatory active substance can retain its pharmaceutically effective dosage but can have remarkably improved toxicity of the tubulin polymerization-inhibitory active substance and an increased the lethal dosage so that its safety zone can be expanded.

The resultant expansion of the safety zone eases the administration burden on patients, as well as application by medical doctors and others in the therapy, betterment, etc., of tumors.

In addition, the present invention also provides the followings:

i) A method for the anti-tumor, wherein the method includes methods for a medical treatment and an improvement of tumors, a prevention of progression of tumor, and a prevention of tumor;

ii) Uses of the above-mentioned 2 effective components for a medical product such as an anti-tumor agent; and

iii) A combination of the above-mentioned 2 effective components wherein the two components are used as a medical product such as an anti-tumor agent, simultaneously or separately.

Accordingly, the present invention can be carried out in the field of medical products and the like, and therefore is very useful industrially.

Numerous modifications and variations on the present invention are possible in light of the above teachings. It is, therefore, to be understood that within the scope of the accompanying claims, the invention may be practiced otherwise than as specifically described herein.

What we claim is:

1. A method for the treatment of tumors wherein the lethal dose of (Z)—N-[2-methoxy-5-[2-(3,4,5-trimethoxyphenyl)vinyl]phenyl]-L-serinamide is increased to twice or more, the toxicity at the pharmaceutically effective dosage of (Z)—N-[2-methoxy-5-[2-(3,4,5-trimethoxyphenyl)vinyl]phenyl]-L-serinamide is reduced, gastrointestinal toxicity at the pharmaceutically effective dosage of (Z)—N-[2-methoxy-5-[2-(3,4,5-trimethoxyphenyl)vinyl]phenyl]-L-serinamide is reduced, hepatic toxicity at the pharmaceutically effective dosage of (Z)—N-[2-methoxy-5-[2-(3,4,5-trimethoxyphenyl)vinyl]phenyl]-L-serinamide is reduced, and/or cardiovascular toxicity at the pharmaceutically effective dosage of (Z)—N-[2-methoxy-5-[2-(3,4,5-trimethoxyphenyl)vinyl]phenyl]-L-serinamide is reduced, which comprises administering to a subject in need thereof a composition comprising
(a) an effective amount of an anti-inflammatory active substance, wherein the anti-inflammatory active substance is a Dexamethasone selected from the group consisting Dexamethasone, an ester of Dexamethasone, and a salt of Dexamethasone; and
(b) (Z)—N-[2-methoxy-5-[2-(3,4,5-trimethoxyphenyl)vinyl]phenyl]-L-serinamide or a salt thereof.

2. The method according to claim 1, wherein said subject in need thereof is a human.

3. The method according to claim 1, wherein said effective amount of said (Z)—N-[2-methoxy-5-[2-(3,4,5-trimethoxyphenyl)vinyl]phenyl]-L-serinamide or a salt thereof ranges from 0.1-10000 mg per day.

4. The method according to claim 1, wherein said effective amount of said anti-inflammatory active substance ranges from 0.1-10000 mg per day.

5. The method according to claim 1, wherein (a) and (b) are administered simultaneously.

6. The method according to claim 1, wherein (a) and (b) are administered sequentially.

7. The method according to claim 1, wherein (a) is Dexamethasone.

8. The method according to claim 1, wherein (a) is an ester of Dexamethasone.

9. The method according to claim 1, wherein (a) is a salt of Dexamethasone.

10. The method according to claim 1, wherein (b) is (Z)—N-[2-methoxy-5-[2-(3,4,5-trimethoxyphenyl)vinyl]phenyl]-L-serinamide.

11. The method according to claim 1, wherein (b) is a salt of (Z)—N-[2-methoxy-5-[2-(3,4,5-trimethoxyphenyl)vinyl]phenyl]-L-serinamide.

12. The method according to claim 1, wherein said method increases the lethal dose of (Z)—N-[2-methoxy-5-[2-(3,4,5-trimethoxyphenyl)vinyl]phenyl]-L-serinamide to twice or more.

13. The method according to claim 1, wherein said method reduces the toxicity at the pharmaceutically effective dosage of (Z)—N-[2-methoxy-5-[2-(3,4,5-trimethoxyphenyevinyl]phenyl]-L-serinamide.

14. The method according to claim 1, wherein said method reduces gastrointestinal toxicity at the pharmaceutically effective dosage of (Z)—N-[2-methoxy-5-[2-(3,4,5-trimethoxyphenyl)vinyl]phenyl]-L-serinamide.

15. The method according to claim 14, wherein said gastrointestinal toxicity is diarrhea.

16. The method according to claim 1, wherein said method reduces hepatic toxicity at the pharmaceutically effective dosage of (Z)—N-[2-methoxy-5-[2-(3,4,5-trimethoxyphenyl)vinyl]phenyl]-L-serinamide.

17. The method according to claim 16, wherein said reducing hepatic toxicity is lowering of GPT.

18. The method according to claim 1, wherein said method reduces cardiovascular toxicity at the pharmaceutically effective dosage of (Z)—N-[2-methoxy-5-[2-(3,4,5-trimethoxyphenyl)vinyl]phenyl]-L-serinamide.

19. The method according to claim 18, wherein said reducing cardiovascular toxicity is lowering of CPK.
